(19) United States
(12) Plant Patent Application Publication
(10) Pub. No.: US 2008/0141413 P1
(43) Pub. Date: Jun. 12, 2008

(54) Geranium plant named 'Zonamaga'

(75) Inventor: Theodorus Cornelis Maria van Kleinwee, Hoorn (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/602,005

(22) Filed: Nov. 20, 2006

(51) Int. Cl. A01H 5/00

(52) U.S. Cl. PLT/327

(57) ABSTRACT

A new Geranium plant, particularly distinguished by the violet flower color, a strong zone, medium vigor and round plants with rather good branching.

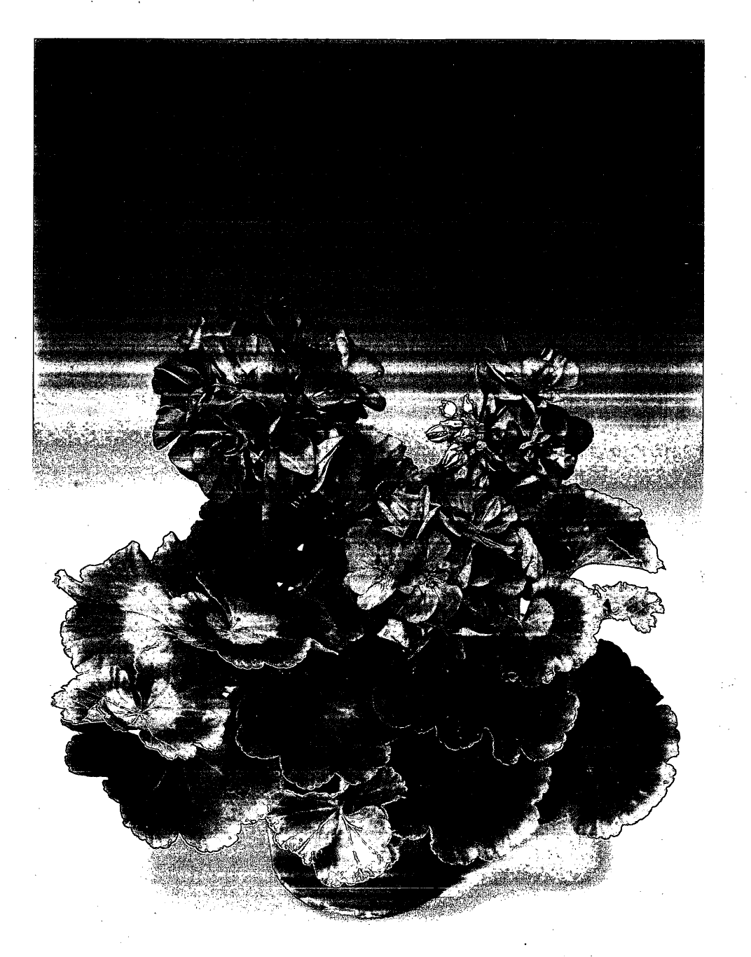

### LATIN NAME OF THE GENUS AND SPECIES OF THE PLANT CLAIMED

Pelargonium zonale (L.) L'Hérit. ex Ait

### VARIETAL DENOMINATION

‘Zonamaga’

### BACKGROUND OF THE NEW PLANT

The present invention comprises a new Geranium, botanically known as Pelargonium zonale (L.) L'Hérit. ex Ait, and hereinafter referred to by the variety name ‘Zonamaga.’

‘Zonamaga’ is a product of a planned breeding program. The new cultivar ‘Zonamaga’ has large, semi-double flowers, is relatively vigorous, but with a well-branched growth habit, and has good heat resistance.

‘Zonamaga’ originates from a hybridization in a controlled breeding program in Enkhuizen, The Netherlands. The female parent was an unpatented hybrid seedling identified as ‘H2182-1’ with scarlet red color, very well branching, and big umbels. The male parent of ‘Zonamaga’ was an unpatented hybrid seedling identified as ‘T010’ with dark salmon flower color, dark green foliage, good branching, and large umbel.

‘Zonamaga’ was selected as one flowering plant within the progeny of the stated cross in 1999 in a controlled environment in Enkhuizen, The Netherlands.

The first act of asexual reproduction of ‘Zonamaga’ was accomplished when vegetative cuttings were taken from the initial selection in the Fall of 1999 in a controlled environment in Enkhuizen, the Netherlands.

Horticultural examination of plants grown from cuttings of the plant initiated in April of 2000 in Enkhuizen, The Netherlands, and continuing thereafter, has demonstrated that the combination of characteristics as herein disclosed for ‘Zonamaga’ are firmly fixed and are retained through successive generations of asexual reproduction. ‘Zonamaga’ has not been observed under all possible environmental conditions. The phenotype may vary significantly with variations in environment such as temperature, light intensity and day length.

### BRIEF DESCRIPTION OF THE NEW VARIETY

The accompanying photographic drawing shows typical flower and foliage characteristics of ‘Zonamaga’ with colors being as true as possible with an illustration of this type. The photographic drawing shows a flowering potted plant of the new variety.

### DETAILED BOTANICAL DESCRIPTION

The measurements were taken in Enkhuizen, The Netherlands, in August 2005 on plants that were growing in window boxes. Culture of these plants started mid February, 2005 in the greenhouse; plants were transferred to window boxes mid May, 2005.

### BRIEF SUMMARY OF INVENTION

The following observations, measurements, and comparisons describe plants grown outside in Enkhuizen, The Netherlands. The following traits have been repeatedly observed and are determined to be basic characteristics of the new variety. The combination of these characteristics distinguishes this Geranium as a new and distinct variety.

TABLE 1
DIFFERENCES BETWEEN THE NEW VARIETY ‘ZONAMAGA’
AND A SIMILAR VARIETY

| | | ‘Americana Dark Red’ (U.S. |
|---|---|---|
| | ‘Zonamaga’ | Plant Patent No. 9,307) |
| Flower Color | 66A | 46B |
| (Upper side of upper | | |
| petal) | | |
| Leaf zoning | Strong Zoning | Hardly any zoning |
| Flower size | Medium | Very big |

Disease/pest resistance: No disease/pest resistance has been observed to date.

What is claimed is:

1. A new and distinct variety of Geranium plant, substantially as illustrated and described herein.
